(19) United States
(12) Patent Application Publication
(10) Pub. No.: US 2011/0086773 A1
(43) Pub. Date: Apr. 14, 2011

(54) DIAGNOSTIC METHODS FOR ORAL CANCER

(75) Inventors: GREGORY ANTON ENDRESS, BELCHERTOWN, MA (US); MADHVI UPENDER, POTOMAC, MD (US); ELIZABETH LIGHT, GAITHERSBURG, MD (US); COLYN CARGILE CAIN, BETHESDA, MD (US)

(73) Assignee: NEODIAGNOSTIX, INC., ROCKVILLE, MD (US)

(21) Appl. No.: 12/900,996

(22) Filed: Oct. 8, 2010

Related U.S. Application Data
(60) Provisional application No. 61/249,720, filed on Oct. 8, 2009.

(51) Int. Cl. C40B 30/04; C12Q 1/68

(52) U.S. Cl. 506/9; 435/6

(57) ABSTRACT

The invention provides for a diagnostic test to monitor cancer-specific genetic abnormalities to diagnose oral cell disorders and predict which patients might progress to cancer. Genetic abnormalities are detected by identification in chromosomal copy number of chromosome 3, chromosome 5 and chromosome 6 using FISH analysis of probes targeted to 3q and/or 5p and/or 11q.

### CROSS REFERENCE TO RELATED APPLICATIONS

This invention claims priority to U.S. Provisional Application No. 61/249,720 filed on Oct. 8, 2009, which is incorporated herein by reference in its entirety and the references cited therein are also incorporated in their entirety by reference herein.

### FIELD

The invention relates to methods and kits for the diagnosis of oral cancer.

### BACKGROUND

Oral cancer is a common cancer worldwide. If detected early, oral cancer and precursor lesions can be treated effectively. Oral squamous cell carcinoma (OSCC) is the most common cancer of the head and neck and accounts for over 300,000 new cases per year worldwide, with 30,000 cases within the United States. The 5-year survival rate has remained at around 50% for the past 20 years, even with advancements in treatment options. One major contributing factor for this is that a majority of oral cancers are not diagnosed or treated until they reach an advanced stage. If treated early, OSCC has an up to 80% 5-year survival rate. It is generally believed that most OSCC develop from premalignant lesions, although, only a small percentage of these premalignant lesions actually progress to carcinoma. In the last 30 years, the rate of oral cancer has increased 15% and continues to be on the rise. Due to the current lack of effective methods to detect and diagnose oral dyplasia, there is a great need for biomarkers to identify premalignant lesions with a high-risk of progression. Recent research studies have reported the key role of chromosomal aneuploidy in the process of OSCC tumorigenesis. Other research studies have implicated copy number variations of specific chromosomal regions as an early stage marker and indicator of disease prognosis. Specifically, these publications have identified chromosomal loci 3q, 5p, and 11q as regions of interest due to the high frequency of their amplification in OSCC.

The majority of oral cancers are not diagnosed or treated until they reach an advanced stage, and thus have a poor prognosis, one of the first indications being oral leukoplakia. If treated early, oral cancer has an up to 80% five-year survival rate. In the last 30 years, the rate of oral cancer has increased 15%, yet there remains a lack of effective methods to detect and diagnose pre-malignant cancers.

Oral leukoplakia is often an indicator of risk for oral cancer. Oral leukoplakia is a white plaque of questionable risk having excluded other diseases or disorders that carry no increased risk or cancer affecting any site of the oral or oropharyngeal cavity. Twenty in 100,000 individuals per year with oral leukoplakia develop oral cancer. Leukoplakia is six times more common in smokers than non-smokers and alcohol is an independent risk factor. (van der Waal, 2009)

There is an histopathological distinction between dysplastic and non-dysplastic leukoplakia. The assessment and severity of dysplasia is based on architectural disturbance and cytological atypia of cells. Architectural disturbances include irregular epithelial stratification, loss of polarity of basal cells, drop-shaped rete ridges, increased number of mitotic figures, abnormal superficial mitoses, premature keratinization in single cells, Keratin pearls with rete pegs. Cytological abnormalities include anisonucleosis, nuclear pleomorphism, anisocytosis, cellular pleomorphism, increased nuclear-cytoplasmic ratio, increased nuclear size, atypical mitotic figures, increased number and size of nucleoli and hyperchromasia. (van der Waal, 2009).

Oral premalignant lesions can be of various types with different levels of malignancy potential. Oral leukoplakia is the most common premalignant lesion of the oral cavity and is defined as a white patch or plaque that cannot be removed. Leukoplakia is not attributable to a specific cause and requires a biopsy for histological evaluation. Estimates of the prevalence of leukoplakia in the general population vary from less than 1% up to 5%, with approximately 2-3% of these lesions developing into carcinoma (Kademani, 2007). Many research studies have analyzed OSCC tumors or tumor derived cell lines by CGH (comparative genomic hybridization) and conventional cytogenetics. These studies have reported a number of recurrent and specific genetic alterations in cells of early dysplastic lesions (leukoplakias) as well as carcinomas (Martin et al, 2008; others).

Current screening practice for oral cancer comprises the identification of suspicious oral lesions or patches by the dentist during a visual exam followed by a biopsy, and diagnosis by a pathologist based on morphological criteria, by cytology or tissue biopsy. There are inherent problems in the morphological analysis of cells, including low sensitivity, subjectivity of interpretation, inter-observer errors. In addition, the progression potential of individual lesions cannot be established. Furthermore, histological grading is valuable for assessment of risk of progression, but limited due to inter and intra-observer variability. (Bremmer et al., 2009). While there are a variety of screening devices that assist doctors in detecting oral cancer, including the Velscope, Vizilite Plus and the identafi 3000, the only definitive method for determining this is through biopsy and microscopic evaluation of the cells in the removed sample. A tissue biopsy, whether of the tongue or other oral tissues, and microscopic examination of the lesion confirm the diagnosis of oral cancer.

Human Papilloma Virus, (HPV) particularly version 16 is a known risk factor and independent causative factor for oral cancer. Gilsion et al. A fast growing segment of those diagnosed does not present with the historic stereotypical demographics. Research suggests that HPV is the primary risk factor in this new population of oral cancer victims. HPV16, along with HPV18, is the same virus responsible for the vast majority of all cervical cancers and is the most common sexually transmitted infection in the US. Oral cancer in this group tends to favor the tonsil and tonsillar pillars, base of the tongue, and the oropharnyx. Recent data suggests that individuals that contract the disease from this particular etiology have some slight survival advantage. (Gilsion, et al).

Presently, uniform reporting of dysplasia and oral cancer recommends the use of a classification and staging system in which the size and the histopathological features of cells are taken into account in addition to age, gender, and time of diagnosis. There are no significant diagnostic tests or biomarkers that can predict the risk of progression for a potentially malignant oral dysplasia to oral squamous cell carcinoma (OSCC).

Therefore, there is a continuing unmet need for molecular analysis of suspicious cells and the definitive diagnosis of abnormalities, because molecular changes are present in cells before phenotypic, cytologic and histologic changes are apparent.

### SUMMARY

The invention provides for a diagnostic method to monitor genetic changes in oral cells using various cytological methods for detecting hybridization using FISH, CISH, flow cytometry, or other methods as are known to those of skill in the art and for detecting genetic abnormalities to predict which patients might progress to cancer and those unlikely to progress, months, if not years, before traditional symptoms present.

The present invention provides for a methodology for a four (4)-color biomarker panel used on oral tissue specimens with or without confirming cytology. The visualization of chromosomal aneuploidy and copy number changes of specific cancer-associated genes can be a stand-alone assessment or an important complement to routine morphological assessment of cytological samples. This approach is biologically valid and successful because chromosomal aneuploidy and the resulting genomic imbalances are specific for cancer cells, distinct for different carcinomas, and occur early during disease progression. Like most other human carcinomas, oral cancers are defined by a distribution of genomic imbalances. The sequential transformation oral epithelial cells to OSCC includes the acquisition of additional copies of chromosome arm 3q, 5p and 11q, among other cytogenetic abnormalities. Identification of 3q26 amplification, amplification of 5p15, and amplification of 11q13 provides information regarding the presence of oral cancer or progressive potential of a lesion.

In one aspect, the present invention provides a method for assessing a patient condition of oral cell disorder which may include OSCC or cancer comprising: detecting, in a sample from a patient: a genomic amplification in chromosome 3q; a genomic amplification in chromosome 5p; a genomic amplification in chromosome 11q and the presence and/or amplification of the centromere of chromosome 6 (CEN6) as control. Detecting the genomic amplification of chromosome 3q, chromosome 5p, and chromosome 11q indicates progression of the patient condition to OSCC. Detection of genomic amplification of chromosome 6 measures the general ploidy status of the oral cell. Typically copy number changes in chromosome 6 do not occur during early stage oral carcinogenesis. If genomic amplification of chromosome 6 is present, it indicates aneuploidy, a state associated with advanced pre-cancers and cancers. The method can assess a change of patient condition of low grade oral cancer to a condition of high grade oral cancer based on degree of chromosomal amplification.

It is yet another aspect of the invention to provide for a method for monitoring a shift from a low risk lesion to high risk legions and oral cancer in a patient. In certain aspects of the invention, the methods disclosed involve identifying a patient at risk of developing invasive oral cancer; and assessing maintenance of a patient condition of oral cancer or regression of a patient condition to low grade oral cancer or normal from more advanced stages of cancer or oral cancer.

In a specific aspect, the methods disclosed herein can further comprise, in addition to detecting genetic amplification in chromosome 3q, 5p and 11q, detecting amplification in chromosomes: 1q; 6q; 7p; 7q; 8q; 11q; 12q; 17p; 18p; 19q; 20q; and any combination thereof. According to more specific aspects of the invention, amplification in the 3q26 locus, 5p15 locus, 11q13 locus is detected. In addition to detection of the 3q26 and 5p15 loci, amplification in the following chromosomal loci can be detected: 1q21-31; 7q11-22; 8q24; 9q33-34; 11q21; 12q13-24; 20q12 and any combination thereof.

In another aspect of the invention, probes directed to the chromosomal regions disclosed are provided, and kits for conducting methods of the invention are provided.

These and other aspects of some exemplary embodiments will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments without departing from the spirit thereof.

### DETAILED DESCRIPTION

The present invention is based on the identification of gain in copy number of chromosomal regions associated with oral cancer. Cancer is a genetic disease, and genetic aberrations can be observed in diseased cells. The aberrations can be observed cytologically, by measuring genetic aberrations either as increase or decrease in gene regions. Gene expression differences are measured by biomarker expression and can be a diagnostic indicator of disease state in the cell. Cytological observations are not required. The methods discussed herein can directly identify abnormalities in the DNA of oral cells using fluorescently labeled probes that bind to the aberrant regions in the chromosome. When greater or less than the expected number of signals are observed, a cell sample can be diagnosed as diseased and oral cancer can be diagnosed even before it is observed cytologically. Patients with these abnormalities can have a poor prognosis and can be at high risk to develop more advanced disease.

As used herein, “oral cancer” means any of the following: oral carcinogenesis, oral squamous cell carcinoma (OSCC), oral cancer, pre-cancer, pre-malignant lesion, oral adenocarcinoma, squamous cell carcinoma of the head and neck (SCCHN), any cytological or genetic abnormality of an oral cell, and any disease or disorder of oral cells. Also, “disease,” “cell disorder,” or “disorder” as used herein includes, but is not limited to, any cytological or genetic abnormality of the cell.

“Oral cells” or “oral” mean any cells or tissue of the mouth, oral cavity or buccal cavity, including tonsil and tonsillar pillars, base of the tongue, mucosa, oropharnyx, salivary glands, gingiva, epithelim lining of the mouth and nose, epiglotis, larynx, esophogous, and/or facial bones of the skull.

The present method provides direct identification of genetic abnormalities in morphologically normal cells and abnormal cells, as well as prognostic information about disease progression, and the flexibility to work with oral cells from any source.

As used herein, “label” or “labels” is any composition, e.g. probe, detectable by spectroscopic, photochemical, biochemical, immunochemical, or chemical means including, but not limited to, fluorescent dyes (e.g. fluorescein, rhodamine, Texas Red, etc., enzymes, electron dense reagents, magnetic labels, and the like). Labels which are not directly detected but are detected through the use of indirect label include biotin and dioxigenin as well as haptens and proteins, for which labeled antisera or monoclonal antibodies are available. Methods of labeling nucleic acids and probes are well known to those of skill in the art. Preferred labels are those that are suitable for use in in situ hybridization. The nucleic acid probes may be detectably labeled prior to hybridization. Alternatively, a detectable label which binds to the hybridization product may be used. Such detectable labels include any material having a detectable physical or chemical property and are well developed in the field of immunoassays.

As used herein “probes” are short nucleic acid sequences of about 15 to about 300 or more nucleic acids in length directed to a portion of a gene on the chromosomal region of interest to detect chromosomal amplification or gain by detection of a label linked to the probe.

### Copy Number Gains

An increase in 3q copy number has been associated with oral cancer, e.g., OSCC and SCCHN. Martin, et al. Higher staged tumors had more chromosomal imbalances including gains of 3q; 8q; and 20q and losses of 3q; 11q; and 9q. 11q gains seen in SCCHN, specifically gain in 11q13 appear to be associated with a tumor site. (Martin, et al.)

Genomic amplification of chromosome regions 3q, 5p and 11q has been reported in a number of studies (Bockmuhl et al., 1996; Martin et al., 2008; Ha et al., 2009). Current literature indicates the amplification of the 3q, 5p, and 11q are the most frequent abnormalities observed in oral cancers and occur at a rate of 40%, 35%, and 45%, respectively. Published data shows these regions are amplified in dysplasia and pre-malignant lesions, but the rate of abnormalities seen varies due to sample sizes and variation in tissue origin. This biomarker panel can be used for early detection of oral squamous cell carcinoma and determine patient prognosis. It is an embodiment of the invention that the timing and incidence of chromosomal amplification at 3q26, 5p15, and 11q13 can be used in diagnostic and prognostic methods for oral cancer and pre-malignant lesions.

The present methods provide for identification or diagnosis of possible oral cancer by comparing the copy number increase of the target chromosomes, for example, 5p, 3q or 11q together, as compared to normal. As used herein, “normal” means chromosomal diploidy in mammalian cells, except when cells that are normally diploid are tetraphase and in the cell cycle, and tetraploidy is observed.

All cells have a normal complement of 23 pairs of chromosomes, a state that is described as diploid. However, when cells grow and undergo cell division, they generate a second set of 23 pairs of chromosomes; one set will subsequently reside in the two daughter cells that are created. This state is described as tetraploid. While tetraploidy is a natural process that occurs throughout the body's tissues and organs on a regular basis, it occurs at low frequency, in general. One hallmark of cancer is uncontrollable cell growth and replication. This typically occurs due to multiple abnormalities in the chromosomes of the cell that enable the cell to escape the standard replication control systems within normal cells. These multiple abnormalities within the chromosomes lead to a state described as aneuploidy, where the chromosome complement is no longer 23 pairs, but something else. Typically, aneuploid cells have extra copies of some chromosomes, have lost other chromosomes, and have even created hybrid chromosomes by fusing two or more together. Very active cell division and tetraploidy provides a foundation for aneuploid cells to develop. Tetraploidy can, therefore, be a transitory condition that indicates a higher risk level for the development of aneuploid cells and more severe cell disorders. Therefore, these methods can measure tetraploidy and provide for the identification of oral cell disorders and possible progression to oral cancer according to the methods disclosed herein.

### Methods

The methods can be used as a diagnostic and prognostic marker for oral cancer. Patients with increased 3q, 5p and 11q copy numbers have a poor prognosis and are at high risk to develop advanced disease.

It is an embodiment of the present invention to identify changes in DNA content and 3q, 5p and 11q copy number in oral cytology samples using multicolor FISH probes directed to loci on chromosomes 3q, 5p and 11q and directed to CEN6, more specifically, the probes are directed to 3q26, 5p15 and 11q13. In a preferred embodiment, probes to different targets will fluoresce with a different color so that targets can be differentiated.

It is an embodiment of the present invention to provide a method for assessing a patient condition of oral cancer, comprising, detecting in a sample from a patient: a genomic amplification in chromosome 3q; a genomic amplification in chromosome 5p; a genomic amplification of 11q and the presence or amplification of CEN6. Detecting the genomic amplification wherein increased copy number of any one of the chromosome regions indicates progression of the patient condition from low grade to high grade oral dysplasia. Moreover, the increased presence of multiple copies increase in expression, thereby indicating more advanced disease.

The methods of the invention can be used to monitor a shift from a low grade to a condition of high grade oral cancer in a patient sample.

The methods disclosed herein may further comprise, in addition to detecting genomic amplification in chromosome 3q and 5p, detecting amplification in chromosomes: 1q; 2q; 6q; 7p; 7q; 8q; 9p; 9q; 10q; 11q; 12q; 16q; 17p; 18p; 19q; 20q; and any combination thereof.

According to specific embodiments of the invention, amplification in the 3q26 locus, 5p15 locus, and 3q13 locus band can be detected. In yet further specific embodiments of the invention, in addition to detection the 3q26, 5p15 and 3q13 loci, amplification in the following chromosomal loci can be detected: 1q21-31; 7q11-22; 8q24; 9q33-34; 11q21; 12g13-24; 20q12; and any combination thereof.

The methods disclosed herein can identify cells or lesions at high risk for oral cancer progression or low risk for oral cancer polygenesis based on progressive chromosomal copy number gain, where risk refers to the increased chance of developing cancer as compared to normal or non-leukoplakial cells.

The methods may be used for assessing and monitoring early stage oral cancer comprising detecting genomic amplification in chromosomes 3q, 5p and 11q. Gain of 11q copy number, gain in 5p copy number and gain in 3q can be severe negative prognostic indicators where gain is observed in the absence of a tumor or in the absence of leukoplakia.

The methods further provide for a specific probe panels including probes to chromosomes 3q, 5p and 11q, and can further include probes to: 1q; 2q; 6q; 7p; 7q; 8q; 9p; 9q; 10q; 11q; 12q; 16q; 17p; 18p; 19q; 20q and any combination of probes thereof. According to specific embodiments of the aforementioned probe panel, probes to the 3q26 locus, 5p15 locus, and 11q13 locus, along or in addition to, probes to at least one of the following chromosomal loci: 1q21-31; 7q11-22; 8q24; 9q33-34; 11q21; 12q13-24; 20q12 and any combination thereof.

The methods and kits disclosed herein may comprise nucleic acid probes targeting genes, including but not limited to CCDN1 (PRADI), EMS1, FGF3, FGF4, TERT, TERC, TRIPI3, PA0S Cri du Chat regions.

One of skill in the art can prepare nucleic acid probes that are complimentary to the sequences of the loci described herein. Additionally, many such probes are commercially available.

### Probes

A number of methods can be used to identify probes which hybridize specifically to the specific loci exemplified herein. For instance, probes can be generated by the random selection of clones from a chromosome specific library, and then mapped by digital imaging microscopy. This procedure is described in U.S. Pat. No. 5,472,842. Various libraries spanning entire chromosomes are also available commercially from for instance Illumina Inc. Probes that hybridize specific chromosomal loci are available commercially from Abbot Molecular, Inc. (Des Plaines, Ill.)

Briefly, a genomic or chromosome specific DNA is digested with restriction enzymes or mechanically sheared to give DNA sequences of at least about 20 kb and more preferably about 40 kb to 300 kb. Techniques of partial sequence digestion are well known in the art. See, for example Perbal, A Practical Guide to Molecular Cloning, 2nd Ed., Wiley N.Y. (1998). The resulting sequences are ligated with a vector and introduced into the appropriate host. Exemplary vectors suitable for this purpose include cosmids, yeast artificial chromosomes (YACs), bacterial artificial chromosomes (BACs) and P1 phage. Various libraries spanning entire chromosomes are also available commercially from, for instance, Genome Systems.

Once a probe library is constructed, a subset of the probes is physically mapped on the selected chromosome. FISH and digital image analysis can be used to localize clones along the desired chromosome. Briefly, the clones are mapped by FISH to metaphase spreads from normal cells using e.g., FITC as the fluorophore. The chromosomes may be counterstained by a stain which stains DNA irrespective of base composition (e.g., DAPI or propidium iodide), to define the outlining of the chromosome. The stained metaphases are imaged in a fluorescence microscope with a polychromatic beam-splitter to avoid color-dependant image shifts. The different color images are acquired with a CCD camera and the digitized images are stored in a computer. A computer program is then used to calculate the chromosome axis, project the two (for single copy sequences) FITC signals perpendicularly onto this axis, and calculate the average fractional length from a defined position, typically the p-telomere. This approach is described, for instance, in U.S. Pat. No. 5,472,842, herein incorporated by reference in its entirety.

Sequence information of the genes identified here permits the design of highly specific hybridization probes or amplification primers suitable for detection of target sequences from these genes. As noted above, the complete sequence of these genes is known. Means for detecting specific DNA sequences within genes are well known to those of skill in the art. For instance, oligonucleotide probes chosen to be complementary to a selected subsequence within the gene can be used. Alternatively, sequences or subsequences may be amplified by a variety of DNA amplification techniques (for example via polymerase chain reaction, ligase chain reaction, transcription amplification, etc.) prior to detection using a probe. Amplification of DNA increases sensitivity of the assay by providing more copies of possible target subsequences. In addition, by using labeled primers in the amplification process, the DNA sequences may be labeled as they are amplified.

### Fluorescent In Situ Hybridization

In one embodiment, probes of the present invention may be directed to at least a portion of TERC gene at band 3q26.2 and TERT or TRIP13 at 5p15.3 and CCDN, EMS1, FGF3, FGF4 or PAOS or at 11q13. Specifically, a probe to TERC at region 3q26 of approximately 495 kb can be used labeled with spectrum gold and also a probe for 5p15 labeled with spectrum green, by way of example, not limitation. Such probes are commercially available from Abbot Molecular (Des Plaines, Ill.). However, the probes of the invention can include any gene on the 3q26 and 5p15 including those listed in FIGS. 1A-D and any combination or portion of the genes on 3q26 or 5p15.

In a specific embodiment, the detectable marker of the probe can emit a fluorescent signal or the probe may be chromogenic. The probes are hybridized using fluorescent in situ hybridization (FISH). FISH is a cytogenetic technique used to detect or localize the presence or absence of specific DNA sequences on chromosomes. FISH uses fluorescent probes that bind to parts of the chromosome with which they show a high degree of sequence similarity. Fluorescence microscopy can be used to find out where the fluorescent probe binds to the chromosome. In situ hybridization is a technique that allows the visualization of specific nucleic acid sequences within a cellular preparation. Specifically, FISH involves the precise annealing of a single stranded fluorescently labeled DNA probe to complementary target sequences. The hybridization of the probe with the cellular DNA site is visible by direct detection using fluorescence microscopy. In instances where additional genetic material is required for testing, the genome may be amplified or detected by Polymerase Chain Reaction (PCR).

It is yet another embodiment of the invention to provide for a procedure of performing FISH on cytology specimens from cheek swabs for successful hybridization of DNA probes in practicing the methods disclosed herein.

It is yet another aspect of the invention to use antibodies to separate squamous and glandular cells out of liquid-based cytology specimens prior to detecting genetic amplification in sample cells. The separation of cell types can improve detection of both squamous and glandular cancers and improve detection of oral carcinomas which are rarely detected through traditional Pap testing but show 3q26 amplification, 5p15 amplification, or both.

The present methods can utilize probes that are fluorescently labeled nucleic acid probes for use in in situ hybridization assays. The labeled probe panel may consist at least of a three-color, three-probe mixture of DNA probe sequences homologous to specific regions on chromosomes 3, 5 and 11; and, as well as other chromosome regions disclosed herein.

It is yet another embodiment of the present methods whereby squamous and/or glandular oral cells can be used from a patient sample to assess chromosomal abnormalities using the present methods.

Typically, it is desirable to use multiple color, in a preferred embodiment three-color FISH methods for detecting chromosomal abnormalities in which three probes are utilized, each labeled by a different fluorescent dye. In the preferred embodiment, two test probes that hybridizes to the regions of interest are labeled with two different dyes and a control probe that hybridizes to a different region which is labeled with a third dye. More than three probes can be used so long as each probe is labeled with a unique dye. A nucleic acid probe that hybridizes to a stable region of the chromosome of interest, such as the centromere, is preferred as a control probe so that differences between efficiency of hybridization from sample to sample can be determined.

Cells recovered and isolated from specimens or samples collected from patients can be fixed on slides. Specimens can be retrieved using various techniques known in the art. In one embodiment specimens can be retrieved from samples.

The samples may also comprise analysis of tissue from oral biopsies, punch biopsies, surgical procedures including but not limited to maxillectomy, mandibulectomy, glossectomy (total, hemi or partial), radical neck dissection or Moh's procedure, combinational procedures e.g. glossectomy and laryngectomy done together. The sample may be prepared from tissue or cells removed from the mouth, head or neck.

### Hybridization

In an embodiment, the regions disclosed here are identified using in situ hybridization. Generally, in situ hybridization comprises the following major steps: (1) fixation of tissue or biological structure to be analyzed; (2) pre-hybridization treatment of the biological structure to increase accessibility of target DNA, and to reduce nonspecific binding; (3) hybridization of the mixture of nucleic acids to the nucleic acid of the biological sample or tissue; (4) post-hybridization washes to remove nucleic acid fragments not bound in the hybridization and (5) detection of the hybridized nucleic acids. Hybridization protocols for the applications described herein are described in U.S. Pat. No. 6,277,563, incorporated herein by reference in its entirety.

From samples, the target DNA can be denatured to its single stranded form and subsequently allowed to hybridize with the probes of the method. Following hybridization, the unbound probe is removed by a series of washes, and the nuclei are counterstained with DAPI (4, 6 diamidino-2-phenylindole), a DNA-specific stain. Hybridization of the DNA probes can be viewed using a fluorescence microscope equipped with appropriate excitation and emission filters allowing visualization of the aqua and gold fluorescent signals. Enumeration of CEN6, 3q26, 5p15 and 11q13 signals is conducted by microscopic examination of the nuclei.

The clinical test disclosed herein can use several biomarkers in combination for the early detection of oral cancer and is important because current morphology based screening and detection methods have significant limitations. Identification of 3q26 and 5p15, among others, amplification and other cytogenetic abnormalities can more precisely and accurately identify patients at risk for developing oral cancer and help them receive earlier treatment.

### Image Analysis

It is an embodiment of the present invention to provide for automatic image analysis and scoring of the methods disclosed. In situ hybridization is a technique that allows the visualization of specific nucleic acid sequences within a cellular preparation. Specifically, DNA fluorescence in situ hybridization (FISH) involves the precise annealing of a single stranded fluorescently labeled DNA probe to complementary target sequences. The hybridization of the probe with the cellular DNA site is visible by direct detection using fluorescence microscopy. The method, as described herein, utilizes probes that are fluorescently labeled nucleic acid probes for use as part of in situ hybridization assays. In a preferred embodiment, the probe panel consists of a 3-color, three-probe mixture of DNA probe sequences homologous to specific regions on chromosomes 3, 5 and 11. The probe mixture consists of a locus specific probe for chromosome 3q26, 5p15, and 11q13 and centromere of CEN6.

It is an embodiment of the present invention to provide for automated image analysis of the signal from the FISH probe. Microscopes can allow for automated capture of digital images of the field of view within the specimen/slide on the microscopy stage. Such manufacturers include Carl Zeiss, Leica, Nikon and Olympus. Also, the method provides for software platforms for automated image analysis such as microscope-software systems developed by such entities as Ikonisys of Connecticut, Metasystems of Massachusetts and Germany and Bioimagene of California, Bioview of Massachusetts. and Israel, among others. Such automated systems may apply to viewing 3q chromosomes alone or in combination with 5p abnormalities in the patient sample.

Cells recovered from specimens can be fixed on slides. The target DNA is denatured to its single stranded form and subsequently allowed to hybridize with the probes. Following hybridization, the unbound probe can be removed by a series of washes, and the nuclei are counterstained with DAPI (4, 6 diamidino-2-phenylindole), a DNA-specific stain. Hybridization of the probes can be viewed using a fluorescence microscope equipped with appropriate excitation and emission filters allowing visualization of the three fluorescent signals. Enumeration of CEN6, 3q26, 5p15 and 11q13 signals is conducted by automated microscopic examination of the nuclei.

The probe set can be viewed, by way of example only, on an epi-fluorescence microscope Spectrum Aqua (CEN6), Spectrum Gold (locus on 3q26), Spectrum Green (locus on 5p15) and Spectrum Red (locus on 11q13) or other labels and probes as are known in the art and disclosed herein.

Clinical Significance of Slide Analysis Procedure: The method disclosed herein is a direct evaluation of chromosomal copy number at specific loci associated with oral cell disorders. The presence of these genetic abnormalities in oral cancer screening specimens, such as a histological analysis test, long before the development of cancer has implications for the management and treatment of patients.

Determination of chromosomal copy number in at least 800 cells, and preferably 1000 cells, can be a sufficient sampling of each clinical specimen. Less than 800 cells or more than 1000 cells can be utilized in this system. The method and system overcome sampling variations and limitations of slide production methodology. The methods and system are consistent with methods recommended by professional medical organizations (ACMG) to determine the threshold between a specimen with and without chromosomal copy number changes. Wolf, D. J. et al. (2007) Period Guidelines for Fluorescence In Situ Hybridization Testing.

In situ hybridization is a technique that allows the visualization of specific nucleic acid sequences within a cellular preparation. Traditionally, the visualization of probe signals has been performed manually by highly-trained personnel. However, it is possible to adapt current technology to automate the image acquisition and analysis process. Microscopes on the market today, such as those manufactured by Carl Zeiss, Leica, Nikon, and Olympus, allow the user to capture digital images of the field of view within the specimen/slide on the microscopy stage. Some of these manufacturers have software available for the automated acquisition of images from specimens/slide. In addition, several entities (Ikonisys, Metasystems, Bioimagene, Aperio, Ventana, among others) have created software platforms specifically for use in commercial laboratories. Some of these entities have systems that include both a microscopy platform and the automated imaging software, including the Ikoniscope Digital Microscopy System by Ikonisys and Metafer and Metacyte by Metasystems.

The type and source of the specimen to be analyzed directly impacts the analysis process and methodology. Each tissue type has its own biology and structure plus each cancer develops differently with different factors affecting the rate of carcinogenesis. Therefore the present invention provides for several methods for automated image acquisition and analysis of specimens.

In another embodiment, the invention provides for an automated system and method for diagnosis and prognosis of oral cancer which captures an image used alternatively for scoring by (1) identifying the image sample number and recording the image used (2) visualizing the signal colors separately (3) analyzing and recording the signal patterns for individual nuclei, selecting the appropriate nuclei based on the criteria described in preceding paragraph and (4) recording the signal numbers.

The scoring data is analyzed by adding the number of any one of the signals (3q, 5p, 11q or CEN6) and dividing by the total number of nuclei scored. A result greater than 2 can be reported as amplified for the given probe. Images are named by the specimen number and slide number and saved.

The scoring data can be analyzed by calculating the number of any one of the signals (e.g. 3q, 5p, 11q or CEN6) and dividing by the total number of nuclei scored; recording that number in the chart at the top of the Score Sheet. A result greater than 2 recorded and reported as amplified for any given probe.

The system and method can be used in conjunction with specimens in liquid suspension that can be placed onto a microscope slide in an even, monolayer of cells, this includes cytology specimens such as plus any fine-needle aspirate (FNA), sputum, or swab-based collection. This automated method screens the entire area covered by cells on the FISH prepared slide and identifies cellular nuclei. The system then enumerates each probe signal and records the copy number of each probe identified. The system continues its automated scoring of cells and chromosomal copy number within each cell. The system categorizes each cell imaged and counted into a category based upon the copy number of each chromosome identified. A normal cell with two copies of each probe 3q26, 5p15, 11q13 and CEN6 would be placed into a 2, 2, 2, 2 category. Abnormal cells would be identified by their probe signal patterns. For instance, a cell with two copies of the CEN6 probe, 5 copies of the 3q26 probe, 3 copies of the 5p15 probe and 3 copies of the 11q13 probe can be placed in the 2, 5, 3, 3 category. Once all of the imaged cells are categorized, the specimen can be evaluated relative to the positive/negative disease threshold. The method and system further provides for automated verification. Specific cell threshold numbers can vary by specimen type and collection method. In addition, the system can be adapted to reflect biological (cell shape, cell size, DNA content of the nucleus, proximity of cells to each other, cell type, etc.) or disease related differences (number of loci with abnormal number, the number of abnormalities at a locus within a single cell, relationship of an abnormality to survival or treatment response). This method and system can be used on a representative sampling of area covered by cells on the slide instead of the entire area, typically this is performed by imaging multiple fields of view or a path based on cellular density until the minimum imaged cell threshold is met.

In other embodiments, the present invention provides for kits for the detection of chromosomal abnormalities at the regions disclosed. In a preferred embodiment, the kits include one or more probes to the regions described herein and any combination of the disclosed probes. The kits can additionally include instruction materials describing how to use the kit contents in detecting the genetic alterations. The kits may also include one ore more of the following: various labels or labeling agents to facilitate the detection of the probes, reagents for the hybridization including buffers, an interphase spread, bovine serum albumin and other blocking agents including blocking probes, sampling devices including fine needles, swabs, aspirators and the like, positive and negative hybridization controls and other controls as are known in the art.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

What is claimed is:

1) A method for detecting chromosomal abnormalities to determine presence of oral cancer in a sample comprising:
hybridizing a patient sample with a nucleic acid probe which binds to a target nucleic acid sequence on chromosome 5p;
hybridizing a patient sample with a nucleic acid probe which binds to a target nucleic acid sequence on chromosome 11q;
hybridizing the patient sample with a nucleic acid control probe which binds to centromere of chromosome 6 (CEN6); and
detecting the formation of the hybridization complexes on chromosome 5p, 11q and on CEN6, wherein said detection of hybridized complex indicates an increase in chromosomal copy number as compared to a normal cell and correlates to the presence of oral cell disorder.

2) A method for detecting chromosomal abnormalities to determine presence of oral cancer in a sample comprising:
hybridizing a patient sample with a nucleic acid probe which binds to a target nucleic acid sequence on chromosome 3q;
hybridizing a patient sample with a nucleic acid probe which binds to a target nucleic acid sequence on chromosome 11q;
hybridizing the patient sample with a nucleic acid control probe which binds to a sequence at centromere of CEN6; and
detecting the formation of the hybridization complexes on chromosome 3q, 11q and on CEN6, wherein said detection of hybridization complex indicates an increase in chromosomal copy number as compared to a normal cell and correlates to the presence of oral cell disorder.

3) A method for detecting chromosomal abnormalities to determine presence of oral cancer in a sample comprising:
hybridizing a patient sample with a nucleic acid probe which binds to a target nucleic acid sequence on chromosome 3q;
hybridizing a patient sample with a nucleic acid probe which binds to a target nucleic acid on chromosome 5p;
hybridizing a patient sample with a nucleic acid probe which binds to a target nucleic acid on chromosome 11q;
hybridizing the patient sample with a nucleic acid control probe which binds to centromere of CEN6; and
detecting the formation of the hybridization complexes on chromosome 5p, on chromosome 3q, on chromosome 11q and on CEN6, wherein said detection of hybridization complex indicates an increase in chromosomal copy number as compared to a normal cell and correlates to the presence of oral disease.

4) The method of claim 1, wherein said oral cancer comprises one or more selected from the group consisting of oral squamous cell carcinoma.

5) The method of claim 2, wherein said oral cancer comprises one or more selected from the group consisting of squamous cell carcinoma of the head and neck.

6) The method of claim 1, wherein the probe binds to target nucleic acid on 5p15 and 11q13.

7) The method of claim 2, wherein the probe binds to target nucleic acid on 3q27 or at 3q26 and at 11q13.

8) The method of claim 3, wherein the probes bind to target nucleic acid sequences on 3q26, 5p15 and 11q13.

9) The method of claim 3, wherein the probes bind to target nucleic acids on 3q26, 5p15 and 11q13.

10) The method of claim 3, wherein the probes binds to target nucleic acid sequences on 5p15 and 3q26.

11) The method of claim 1 or 3, wherein the target nucleic acid sequence on 5p comprises targets at loci selected from the group consisting of 5p15.33, 5p15.32, 5p15.31, 5p15.2, 5p15.1 or any portion or combination thereof.

12) The method of claims 2 or 3, wherein the target nucleic acid sequence on 3q comprises targets at loci selected from the group consisting of 3q26.1, 3q26.2, 3q26.31, 3q26.32, 3q26.33.

13) The method of claim 1 or 3, wherein the target nucleic acid sequence on chromosome arm 5p comprises a nucleic acid sequence from the TERT gene.

14) The method of claim 2 or 3, wherein the target nucleic acid sequence on chromosome arm 3q comprises nucleic acid sequences from TERC.

15) The method of claim 1 or 3, wherein the target nucleic acid sequence on chromosome arm 5p comprises nucleic acid sequences from TRIP13.

16) The method of claim 2 or 3, wherein the target nucleic acid comprises PIC3CA or GLUT2.

17) The method according to claim 1 or 3, wherein the target nucleic acid sequence is at the Cri du Chat locus at 5p15.

18) The method according to claims 1, 2 or 3, wherein the target nucleic acids on 11q comprise sequences of the Cyclin B5 gene or PAOS gene.

19) The method according to claims 1, 2 or 3, further comprising probes targeted to one or more arms comprising 1q; 2q; 6q; 7p; 7q; 8q; 9p; 9q; 10q; 11q; 12q; 16q; 17p; 18p; 19q; 20q and any combination thereof.

20) The method according to claims 1, 2 or 3, further comprising probes targeted to one or more regions comprising 1q21-31; 7q11-22; 8q24; 9q33-34; 11q21; 12q13-24; 20q12 and any combination thereof.

21) The method of claims 1, 2 or 3, wherein tetraploidy is observed.

22) The method of claims 1, 2 or 3, further comprising detection using FISH, CISH, PCR, ELISA, CGH, Array CGH or flow cytometry.

23) The method of claims 1, 2 or 3, wherein the sample comprises metaphase cells or interphase cells.

24) The method of claims 1, 2 or 3, wherein the sample comprising oral cells is obtained from a fine-needle aspirate (FNA), sputum, or swab-based collection.

25) The method according to claim 1, 2 or 3, wherein the sample is derived from a patient positive for human papilloma virus infection.

26) A kit for detecting chromosomal alterations according to claims 1, 2 or 3.

27) A method for assessing a shift in patient condition comprising the steps of the method according to claims 1, 2 or 3.

28) The method according to claim 23, further comprising determining a shift in patient condition from low grade to high grade oral cancer.

29) A method for determining a patient's risk, predisposition for or likelihood of developing a oral cancer comprising the steps of the method according to claims 1, 2 or 3.

30) A method for assessing maintenance or regression of a patient condition to low grade oral cancer or normal comprising the steps of the method according to claims 1, 2 or 3.

31) The method according to claims 1, 2 or 3, wherein the method detects, confirms or verifies the successful treatment of a oral cancer.

32) The method according to claims 1, 2 or 3, wherein the method quantifies the extent and/or severity of a oral cancer.

33) The method of claims 1, 2 or 3, wherein said detecting further comprises automated imaging analysis of the hybridization complexes.

34) An automated method for detecting chromosomal abnormalities to determine presence of cervical cell disease in a patient sample comprising:
contacting said patient sample comprising cervical cells on a slide with at least two distinguishably labeled probes directed to a portion of a chromosomal region of said cervical cells;
hybridizing a target nucleic acid region on chromosome 5p or chromosome 3q in said cervical cells with a first said labeled nucleic acid probe;
hybridizing a target nucleic acid region on chromosome 11q in said cervical cells with a first said labeled nucleic acid probe;
hybridizing a target nucleic acid region on centromere of chromosome 6 (CEN6) with a second said labeled nucleic acid probe; and
automatically scanning said sample with a microscope to detect the formation of the hybridization complexes on chromosome 5p and on CEN7 and to generate an image after sufficient time and conditions to permit hybridization;
automatically analyzing said image to characterize the chromosomal profile in said cells to generate a diagnosis; and
automatically reporting said diagnosis to a user.

35) A kit for practicing the method of claims 1, 2 or 3 comprising probes to the chromosomal regions selected from 5p, 3q, 11q or CEN6.
